(19) United States
(12) Patent Application Publication
(10) Pub. No.: US 2019/0233844 A1
(43) Pub. Date: Aug. 1, 2019

(54) SPATIOTEMPORAL REGULATORS

(71) Applicant: SENTI BIOSCIENCES, INC., South San Francisco, CA (US)

(72) Inventors: Timothy Kuan-Ta LU, Cambridge, MA (US); Remus WONG, San Francisco, CA (US)

(73) Assignee: Senti Biosciences, Inc., South San Francisco, CA (US)

(21) Appl. No.: 16/319,791

(22) PCT Filed: Jul. 26, 2017
(86) PCT No.: PCT/US2017/043938

§ 371 Date: Jan. 22, 2019

Related U.S. Application Data
(60) Provisional application No. 62/366,755, filed on Jul. 26, 2016.

(51) Int. Cl. C12N 15/86; C12N 15/62; C12N 15/113

(52) U.S. Cl. CPC C12N 15/86; C12N 15/62; C12N 15/113; C12N 2830/30; C12N 2310/141; C12N 2310/122; C12N 2840/105; C12N 2830/008

(57) ABSTRACT

Provided herein, in some embodiments, are methods, compositions, systems and kits that enable spatiotemporal regulation of nucleic acid expression in engineered cells.

### RELATED APPLICATION

This application claims the benefit under 35 U.S.C. § 119(e) of U.S. provisional application No. 62/366,755, filed Jul. 26, 2016, which is incorporated by reference herein in its entirety.

### SUMMARY

Provided herein, in some embodiments, are methods, compositions, systems and kits for the spatiotemporal control of cellular function ex vivo and in vivo. Spatiotemporal regulators of the present disclosure integrate synthetic promoters that enable selective nucleic acid expression in target cells (on-target cells) and microRNA (miRNA) sensors that enable suppression of nucleic acid expression in non-target cells (off-target cells). The synthetic promoters used herein exhibit more accurate specificity relative to naturally-occurring promoters and exhibit higher activity in target cells relative to non-target cells. The miRNA sensors include at least one (one or more) miRNA binding sites specific for miRNAs that are active in non-target cells (leading to suppression/degradation in non-target cells), but are inactive or are active at low levels in target cells. This dual functionality enables enhanced nucleic acid expression selectively in target cells.

This technology is broadly transformative for establishing, engineering, manufacturing, and deploying next-generation human cell therapies. Spatiotemporal control over small molecules or biologic drugs is difficult, making it challenging to treat complex diseases where localization, timing, or dynamics are important. Engineered cells offer the potential for spatiotemporal control of therapies in vivo. The engineered genetic constructs (spatiotemporal regulators) as provided herein may be used for spatiotemporal programming of cell function, enabling spatiotemporal control of therapies in vivo.

Thus, some aspects of the present disclosure provide engineered genetic constructs comprising at least one synthetic promoter that has higher activity in target cells relative to non-target cells and is operably linked to (a) a nucleotide sequence encoding a product of interest and (b) a 3′ untranslated region (UTR) comprising a microRNA (miRNA) sensor that includes at least one miRNA binding site to which at least one miRNA binds, wherein the at least one miRNA is inactive or is active at a low level in the target cells, and wherein the at least one miRNA is active in non-target cells at a level detectable by the miRNA sensor.

### BRIEF DESCRIPTION OF THE DRAWINGS

FIG. 1 shows an example of engineered viral vectors inhibiting the NF-κB pathway selectively in microglia (not in other cell types) may ameliorate neuroinflammation in ALS and other neurological diseases. Genetic constructs are delivered via viral vectors (e.g., AAV) into brain cells and then rely on microglia-specific promoters and microRNA sensors to achieve selective expression of NF-κB inhibitors only in microglia. NF-κB inhibitors can be expressed under the control of NF-κB-induced promoters to enable dynamic repression rather than constitutive repression.

FIG. 2 shows examples of modular constructs used to assess various cell-specific regulators (e.g., promoters, 5′UTRs, 3′UTRs). Synthetic promoters may be combined with synthetic miRNA binding sites in the 3′UTRs, as well as engineered 5′UTRs, in some instances, to assess gene expression for a wide range of applications.

FIG. 3 shows an examples of a microRNA sensor vector used for assaying individual constructs.

FIG. 4, top panel, is a graph of data showing microRNAs inhibiting expression of the reporter gene at different levels for the two cell lines described in Example 1. In each set of bars, left to right: MCF-10A; MDA-MB-453. FIG. 4, bottom panel, is a graph of data showing expression ratios between the cell types, showing that certain microRNAs have greater than 5-fold selectivity between the cell types. In each set of bars, left to right: MDA/MCF; MCF/MDA.

FIG. 5 shows an example of a construct integrating a synthetic promoter and microRNA sensors.

FIG. 6A shows expression data on combinations of synthetic promoters and microRNAs. In each set of bars, left to right: pmirGLO; pSyn-3; pSyn-12; pSyn-18. FIG. 6B is a magnified view of the same data. In each set of bars, left to right: pmirGLO; pSyn-3; pSyn-12; pSyn-18.

FIG. 7 shows additional expression data on combinations of synthetic promoters and microRNAs. In each set of bars, left to right: pmirGLO; pSyn-3; pSyn-12; pSyn-18. FIG. 7B is a magnified view of the same data. In each set of bars, left to right: pmirGLO; pSyn-3; pSyn-12; pSyn-18.

FIGS. 8A-8B shows the ratio of expression data for combinations of synthetic promoters and microRNAs. An unexpected synergistic effect was observed in several of the constructs containing a synthetic promoter and a microRNA sensor. As an example, promoter pSyn-18 alone showed ˜25× selectivity, miR-29A(1×) was ˜4×, and the combination was ˜250×. In each set of bars, left to right: pmirGLO; pSyn-3; pSyn-12; pSyn-18.

### DETAILED DESCRIPTION

Provided herein are engineered genetic constructs that achieve spatial and/or temporal selectivity, enabling improved (enhanced) control over cell function ex vivo and in vivo. These engineered genetic constructs may be referred to as spatiotemporal regulators. Spatiotemporal regulators can be used to create cell and gene therapies that are conditionally activated in specific target cells (e.g., cancer cells such as ovarian cancer cells or microglia) and suppressed in non-target cell types (e.g., non-cancerous cells) to produce therapeutic outputs (e.g., immunotherapies or anti-inflammatory mediators, respectively). In addition, these regulators can be used to create cell and gene therapies that are conditionally activated in certain conditions (e.g., inflammation) and suppressed in other conditions (e.g., non-inflammatory). The ability to localize, concentrate, and time the expression of therapeutic effectors enables the treatment of complex diseases for which dynamics play an important role.

Thus, described herein is a powerful technology that enables next-generation cell and gene therapies. These spatiotemporal regulators can be used to control the expression of genetic constructs in specific cell types, under specific conditions, and/or at specific times. This technology enables the conditional or localized production of therapeutics in vivo and ex vivo. The spatiotemporal regulators leverage a rational design approach combined with high-throughput design-build-test-learn platform to rapidly converge on gene expression constructs that are only activated in cell-type/cell-state-specific fashion. These regulators achieve highly active and specific gene expression in target cells using complementary integrated mechanisms to enhance stringency and activity.

Target specificity in gene therapies is currently achieved primarily through the use of targeted viral vectors or natural promoters. The former is challenging because there is not always a unique cell-surface marker that can be used for specific viral targeting. The latter is challenging because natural promoters not are always completely specific for a certain cell type, can have low ON-OFF ratios in on-target versus off-target cells, and can be quite large in size, thus limiting encoding in viral vectors with restricted capacities. As described herein, >30-fold ON-OFF ratios are achieved in the integrated genetic constructs as minimal targets, and >100-fold ON-OFF ratios are achieved in some embodiments. Such ON-OFF ratios are reasonable given that narrow therapeutic index drugs often have <2-fold differences between minimum effective concentrations versus minimum toxic concentrations (26).

In some embodiments, the synthetic promoters and microRNA sensors can be incorporated into logic gates, such as AND gates, and/or digital switches to set sharper thresholds for gene expression. The output genes may be, for example, therapeutic payloads such as immunotherapy outputs for cancer applications or inhibitors of inflammation for neuroinflammation/neurodegeneration applications (e.g., amyotrophic lateral sclerosis [ALS]).

For example, for cancer applications, the secretion of checkpoint inhibitors, cytokines, and chemokines, and the surface display of anti-CD3c domains to trigger T cells to kill cancer cells may be useful. High stringency is required in order to minimize off-target effects on normal cells. The engineered genetic constructs as provided herein may be delivered to cancer cells via non-viral vectors or viruses to recruit immunotherapy to kill tumors from within the tumors themselves. This approach overcomes major limitations in existing immunotherapies. For example, CAR T cells and bispecific T-cell engagers require specific cell-surface targets that can be difficult to find. Also, tumors can create immunosuppressive environments that are challenging to overcome with conventional approaches.

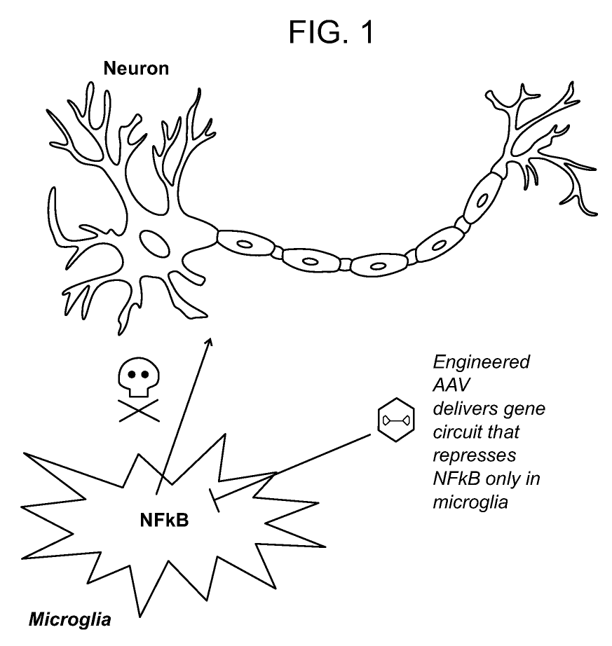

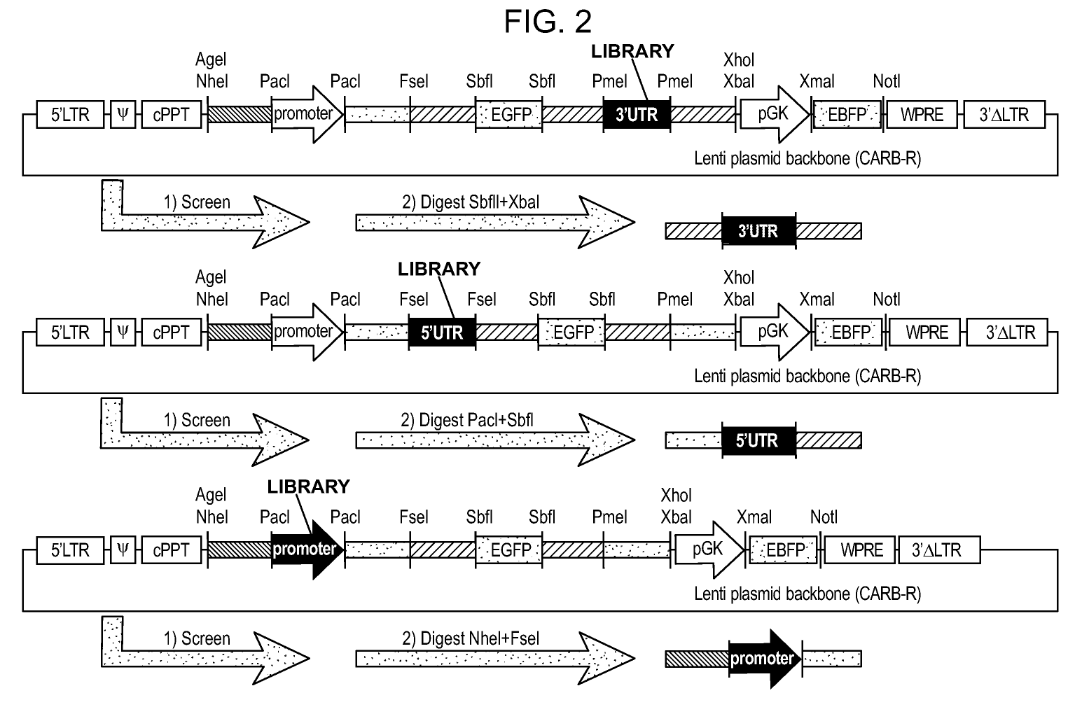

In addition, recent data in ALS models have shown that NF-κB-mediated pathways in microglia result in motor neuron death (1). Global suppression of inflammation does not improve survival of ALS mice and can even exacerbate disease. Furthermore, NF-κB mediates important signaling pathways in neurons and thus suppressing it in an untargeted fashion is likely to be undesirable. However, targeted inhibition of the NF-κB pathway in microglia can extend the lifespan of SOD1-G93A mice, a model of ALS, by up to 47%. Thus, spatial/cell-type-specific inhibition of the NF-κB pathway using the constructs described herein and delivered into the brain via AAV vectors is a transformative approach for treating diseases associated with neuroinflammation, including ALS (FIG. 1). Further temporal control of microglia-specific NF-κB inhibition with switches that are regulated with exogenous FDA-approved drugs or natural products (9-12), enables further regulation over the safety and timing of such approaches.

Some aspects of the present disclosure provide engineered genetic constructs comprising at least one synthetic promoter that has higher activity in target cells relative to non-target cells and is operably linked to (a) a nucleotide sequence encoding a product of interest and (b) a 3′ untranslated region (UTR) comprising a microRNA (miRNA) sensor that includes at least one miRNA binding site to which at least one miRNA binds, wherein the at least one miRNA is inactive or is active at a low level in target cells, and wherein the at least one miRNA is active in non-target cells at a level detectable by the miRNA sensor.

A synthetic promoter is considered to have higher activity in target cells relative to non-target cells if expression of the nucleic acid to which the synthetic promoter is operably linked is higher (e.g., at least 10%) in target cells relative to non-target cells (even in the absence of a miRNA sensor as provided herein). In some embodiments, the activity of a synthetic promoter is at least 10% higher in target cells relative to non-target cells. For example, the activity of a synthetic promoter may be at least 15%, 20%, 25%, 30%, 35%, 40%, 45%, 50%, 55%, 60%, 65%, 70%, 75%, 80%, 85%, 90%, 95%, 100%, 200%, 300%, 400%, 500%, 1000%, 1500%, 2000%, 2500%, 3000%, 3500%, 4000%, 4500%, 5000% or higher in target cells relative to non-target cells. In some embodiments, the activity of a synthetic promoter is 10%-1000%, 10%-500%, 10%-100%, 50%-1000%, 50%-500%, or 50%-100% higher in target cells relative to non-target cells. In some embodiments, the activity of a synthetic promoter is at least 50% higher in target cells relative to non-target cells. In some embodiments, the activity of a synthetic promoter is at least 100% higher in target cells relative to non-target cells.

A miRNA is considered active in non-target cells if the miRNA is present in the non-target cells at a level sufficient to suppress expression of the nucleic acid (e.g., by greater than 10%, 20%, 30%, 40%, 50%, 60%, 70%, 80%, or 90%) to which the synthetic promoter is operably linked. A miRNA is considered inactive in the target cells if the miRNA is absent from the target cells (the target cells are free of the particular miRNA) or if the miRNA does not bind to the miRNA sensor. A miRNA is considered active at a low level the target cells if the mRNA is present in the target cells but not at a level sufficient to suppress expression of the nucleic acid (silence translation or degrade transcript) (e.g., by greater than 10%, 20%, 30%, 40%, 50%, 60%, 70%, 80%, or 90%) to which the synthetic promoter is operably linked.

### Synthetic Promoters

A synthetic promoter of the present disclosure is a control region of a nucleic acid sequence at which initiation and rate of transcription of the remainder of a nucleic acid sequence are controlled. A synthetic promoter typically contains sub-regions at which regulatory proteins and molecules may bind, such as RNA polymerase and other transcription factors. A synthetic promoter drives expression or drives transcription of the nucleic acid sequence that it regulates. A synthetic promoter is considered to be operably linked when it is in a correct functional location and orientation in relation to a nucleic acid sequence it regulates to control (“drive”) transcriptional initiation and/or expression of that sequence.

Synthetic (non-naturally-occurring) promoters of the present disclosure, in some embodiments, have a length of 100-500 nucleotides. For example, a synthetic promoter may have a length of 100, 200, 300, 400 or 500 nucleotides. In some embodiments, a synthetic promoter has a length of 200-300 nucleotides. In some embodiments, a synthetic promoter has a length of 100-125 nucleotides.

In some embodiments, a synthetic promoter includes tandem repeat nucleotide sequences. That is, a synthetic promoter may include repeat (identical) nucleotide sequences located directly adjacent to each other (contiguous with each other), or separated from each other by only a few (e.g., 1-5) nucleotides (by nucleotide spacers). Thus, in some embodiments, a nucleotide spacer having a length of 1-5 nucleotides (e.g., 1, 2, 3, 4 or 5 nucleotides) is positioned between repeat nucleotides sequences. The nucleotide spacers may be selected from AGC, ATC, GAC, ACT, AGT, GTC, GAT, and GCT, for example. In some embodiments, the spacers between repeat nucleotide sequences vary in length (are not the same length relative to each other).

The length of a repeat nucleotide sequence of a synthetic promoter, in some embodiments, is less than 12 nucleotides. For example, the length of a repeat nucleotide sequence may be 11, 10, 9, 8, 7, 6, 5 or 4 nucleotides. In some embodiments, the length of a repeat nucleotide sequence is 4-11, 4-10, 4-9, 4-8, 4-7, 4-6, 5-11, 5-10, 5-9, 5-8, 6-11, 6-10, 6-9, 7-11 nucleotides. In some embodiments, the length of a repeat nucleotide sequence is 11 nucleotides. In some embodiments, the length of a repeat nucleotide sequences is 8 nucleotides. Lengths of greater than 12 nucleotides may also be used.

In some embodiments, the synthetic promoter includes 2-20 tandem repeat nucleotide sequences. For example, a synthetic promoter may include 2, 3, 4, 5, 6, 7, 8, 9, 10, 11, 12, 13 14, 15, 16, 17, 18, 19 or 20 tandem repeat nucleotide sequences. In some embodiments, the synthetic promoter includes 2-15, 2-10, 2-5, 5-10, 5-15, or 5-10 tandem repeat nucleotide sequences.

miRNA Sensors

A microRNA (miRNA) is a small non-coding RNA molecule (e.g., containing about 22 nucleotides) that typically functions in RNA silencing and post-transcriptional regulation of gene expression. miRNA molecules include a sequence wholly or partially complementary to sequences found in the 3′ untranslated region (UTR) of some mRNA transcripts. Binding of a miRNA to a miRNA binding site in the 3′UTR of a mRNA leads to silencing that may occur via mRNA degradation or prevention of translation.

As provided herein, miRNAs identified as downregulated in specific target cells but not in non-target cells are used to suppress the expression of nucleic acids encoding a product of interest (e.g., output gene) in non-target cells that contains miRNA binding sequences in their mRNA sequences. Thus, miRNA-based suppression of gene expression occurs, in some embodiments, only in non-target cells, resulting in reduced gene expression compared with target cells.

miRNA sensors of the present disclosure include at least one or at least two mRNA binding sites to which specific miRNAs bind to silence expression of the nucleic acid to which a synthetic promoter is operably linked. For example, a miRNA sensor may include, at least 3, 4, 5, 6, 7, 8, 9 or 10 miRNA binding sites. In some embodiments, a miRNA sensor includes at least five (or five) miRNA binding sites. In some embodiments, a miRNA sensor includes 1-10 miRNA binding sites. For example, a miRNA sensor may include 1-9, 1-8, 1-7, 1-6, 1-5, 1-4, 1-3, 1-2, 2-9, 2-8, 2-7, 2-6, 2-5, 2-4, 2-3, 3-10, 3-9, 3-8, 3-7, 3-6, 3-5, 3-4, 4-10, 4-9, 4-8, 4-7, 4-6, 4-5, 5-10, 5-9, 5-8, 5-7, 5-6, 6-10, 6-9, 6-8, 6-7, 7-10, 7-9, 7-9, 8-10, 8-9, or 9-10 miRNA binding sites. In some embodiments, a miRNA sensor includes 2-10 miRNA binding sites. In some embodiments, a miRNA sensor includes 5-10 miRNA binding sites.

In some embodiments, the mRNA binding sites are located in tandem. That is, the miRNA binding sites may be directly adjacent to each other (contiguous with each other), or separated from each other by nucleotide spacers (e.g., spacers having lengths of 1-10, e.g., 1, 2, 3, 4, 5, 6, 7, 8, 9 or 10) nucleotides.

In some embodiments, the miRNA binding sites within a single miRNA sensor are identical to each other (have the same nucleotide sequence). In some embodiments, the miRNA binding sites within a single miRNA sensor share at least 80% (e.g., at least 85%, 90%, 95%, 96%, 97%, 98%, or 99%) nucleotide sequence identity.

The length of a miRNA binding site may vary. In some embodiments, the length of a miRNA binding site is 15-30 nucleotides. For example, the length of a miRNA binding site may be 15, 16, 17, 18, 19, 20, 21, 22, 23, 24, 25, 26, 27, 28, 29, or 30 nucleotides. In some embodiments, the length of a miRNA binding site is 15-20, 20-30, or 20-25 nucleotides.

In some embodiments, a miRNA binding site is wholly (100%) complementary to the miRNA, while in other embodiments, a miRNA binding site is partially (less than 100%) complementary to the miRNA.

An engineered genetic construct may include a single miRNA sensor (e.g., comprising one or multiple miRNA binding sites) or multiple (more than one) miRNA sensors. Multiple mRNA binding sites within a single miRNA sensor, or multiple miRNA sensors (e.g., each containing different miRNA binding sites), may be used in combination with synthetic promoters to enhance the stringency of cell-type specificity and cell-state specificity. Thus, in some embodiments, multiple miRNA binding sites within a single miRNA sensor, or multiple different miRNA sensors, can be encoded in tandem on the 3′end of target transcripts so that high-level gene expression is allowed only when multiple microRNAs are inactive (e.g., absent) or have low activity in target cells. In some embodiments, the 3′ UTR comprises at least two (e.g., at least 3, 4 or 5) miRNA sensors, each specific to a different miRNA. In embodiments, wherein a construct includes more than one miRNA sensor, the sensors include miRNA binding sites specific to different miRNAs. For example, an engineered genetic construct may include a first miRNA sensor comprising a single mRNA binding site or tandem repeat miRNA binding sites specific for miRNA#1 (e.g., miR-54), and the same engineered construct may include a second (or more) miRNA sensor comprising a single miRNA binding site or tandem repeat miRNA binding sites specific for miRNA#2 (e.g., miR-497). As demonstrated herein, greater than 3-fold selectivity, and in some instances greater than 5-fold selectivity is achieved using the engineered genetic constructs with multiple mRNA binding sites and/or sensors.

In some embodiments, at least one miRNA (miR) is selected from miR-154, miR-497, miR-29A, miR-720, miR-205, miR-494, miR-224, miR-191, miR-21, miR-96, miR-449A and miR-183.

### Products of Interest

Products encoded by the engineered genetic constructs of the present disclosure may be, for example, therapeutic molecules and/or prophylactic molecules. In some embodiments, the product of interest is protein or peptide (e.g., a therapeutic protein or peptide). In some embodiments, the product of interest is a nucleic acid (e.g., a therapeutic nucleic acid). Examples of nucleic acids include RNA, DNA or a combination of RNA and DNA. In some embodiments the product interest is DNA (e.g., single-stranded DNA or double-stranded DNA). In some embodiments, the product of interest is RNA. For example, the product of interest may be selected form RNA interference (RNAi) molecules, such as short-hairpin RNAs, short interfering RNAs and micro RNAs. In some embodiments, a product of interest controls viral replication and/or virulence.

Examples of therapeutic and/or prophylactic molecules, such as antibodies (e.g., monoclonal or polyclonal; chimeric; humanized; including antibody fragments and antibody derivatives (bispecific, trispecific, scFv, and Fab)), enzymes, hormones, inflammatory molecules, anti-inflammatory molecules, immunomodulatory molecules, and anti-cancer molecules. Specific examples of the foregoing classes of therapeutic molecules are known in the art, any of which may be used in accordance with the present disclosure.

In some embodiments, the product of interest is an immunomodulatory molecule. An immunomodulatory molecule is a molecule (e.g., protein or nucleic acid) that regulates an immune response. In some embodiments, the immunomodulatory molecules are expressed at the surface of, or secreted from, a cancerous cell or secreted from a cancerous cell.

In some embodiments, the immunomodulatory molecule is a synthetic T cell engager (STE). A synthetic T cell engager is a molecule (e.g., protein) that binds to (e.g., through a ligand-receptor binding interaction) a molecule on the surface of a T cell (e.g., a cytotoxic T cell), or otherwise elicits a cytotoxic T cell response. In some embodiments, an STE is a receptor that binds to a ligand on the surface of a T cell. In some embodiments, an STE is an anti-CD3 antibody or antibody fragment. A STE of the present disclosure is typically expressed at the surface of, or secreted from, a cancer cell or other disease cell to which a nucleic acid encoding the STEs is delivered. See, e.g., International Publication Number WO 2016/205737, incorporated herein by reference.

Examples of STEs of the present disclosure include antibodies, antibody fragments and receptors that binds to T cell surface antigens. T cell surface antigens include, for example, CD3, CD4, CD8 and CD45.

In some embodiments, a product of interest is selected from chemokines, cytokines and checkpoint inhibitors.

Immunomodulatory molecule include immunostimulatory molecule and immunoinhibitory molecule. An immunostimulatory molecule is a molecule that stimulates an immune response (including enhancing a pre-existing immune response) in a subject, whether alone or in combination with another molecule. Examples include antigens, adjuvants (e.g., TLR ligands, nucleic acids comprising an unmethylated CpG dinucleotide, single-stranded or double-stranded RNA, flagellin, muramyl dipeptide), cytokines including interleukins (e.g., IL-2, IL-7, IL-15 (or superagonist/mutant forms of these cytokines), IL-12, IFN-gamma, IFN-alpha, GM-CSF, FLT3-ligand, etc.), immunostimulatory antibodies (e.g., anti-CTLA-4, anti-CD28, anti-CD3, or single chain/antibody fragments of these molecules), and the like.

An immunoinhibitory molecule is an molecule that inhibits an immune response in a subject, whether alone or in combination with another molecule. Examples include anti-CD3 antibody or antibody fragment, and other immunosuppressants.

Antigens may be, without limitation, a cancer antigen, a self-antigen, a microbial antigen, an allergen, or an environmental antigen.

A cancer antigen is an antigen that is expressed preferentially by cancer cells (e.g., it is expressed at higher levels in cancer cells than on non-cancer cells) and in some instances it is expressed solely by cancer cells. The cancer antigen may be expressed within a cancer cell or on the surface of the cancer cell. The cancer antigen may be MART-1/Melan-A, gp100, adenosine deaminase-binding protein (ADAbp), FAP, cyclophilin b, colorectal associated antigen (CRC)—C017-1A/GA733, carcinoembryonic antigen (CEA), CAP-1, CAP-2, etv6, AML1, prostate specific antigen (PSA), PSA-1, PSA-2, PSA-3, prostate-specific membrane antigen (PSMA), T cell receptor/CD3-zeta chain, and CD20. The cancer antigen may be selected from the group consisting of MAGE-A1, MAGE-A2, MAGE-A3, MAGE-A4, MAGE-A5, MAGE-A6, MAGE-A7, MAGE-A8, MAGE-A9, MAGE-A10, MAGE-A11, MAGE-A12, MAGE-Xp2 (MAGE-B2), MAGE-Xp3 (MAGE-B3), MAGE-Xp4 (MAGE-B4), MAGE-C1, MAGE-C2, MAGE-C3, MAGE-C4, MAGE-C5). The cancer antigen may be selected from the group consisting of GAGE-1, GAGE-2, GAGE-3, GAGE-4, GAGE-5, GAGE-6, GAGE-7, GAGE-8, GAGE-9. The cancer antigen may be selected from the group consisting of BAGE, RAGE, LAGE-1, NAG, GnT-V, MUM-1, CDK4, tyrosinase, p53, MUC family, HER2/neu, p21ras, RCAS 1, α-fetoprotein, E-cadherin, α-catenin, β-catenin, γ-catenin, p120ctn, gp100Pmel117, PRAME, NY-ESO-1, cdc27, adenomatous polyposis coli protein (APC), fodrin, Connexin 37, Ig-idiotype, p15, gp75, GM2 ganglioside, GD2 ganglioside, human papilloma virus proteins, Smad family of tumor antigens, Imp-1, P1A, EBV-encoded nuclear antigen (EBNA)-1, brain glycogen phosphorylase, SSX-1, SSX-2 (HOM-MEL-40), SSX-1, SSX-4, SSX-5, SCP-1 and CT-7, CD20, and c-erbB-2.

In some embodiments, a product of interest is a diagnostic molecule. The diagnostic molecule may be, for example, a detectable molecule, e.g., detectable by microscopy. In some embodiments, the diagnostic molecule is a fluorescent molecule, such as a fluorescent protein. Fluorescent proteins are known in the art, any of which may be used in accordance with the present disclosure. In some embodiments, the diagnostic molecule is a reporter molecule that can be imaged in a subject (e.g., human subject). For example, the reporter molecule may be a sodium iodide symporter (see, e.g., Galanis, E. et al. Cancer Research, 75(1): 22-30, 2015, incorporated herein by reference).

### Engineered Nucleic Acids

An engineered nucleic acid (e.g., an engineered genetic construct) is a nucleic acid that does not occur in nature. It should be understood, however, that while an engineered nucleic acid as a whole is not naturally-occurring, it may include nucleotide sequences that occur in nature. In some embodiments, an engineered nucleic acid comprises nucleotide sequences from different organisms (e.g., from different species). For example, in some embodiments, an engineered nucleic acid includes a murine nucleotide sequence, a bacterial nucleotide sequence, a human nucleotide sequence, and/or a viral nucleotide sequence. The term “engineered nucleic acids” includes recombinant nucleic acids and synthetic nucleic acids. A “recombinant nucleic acid” refers to a molecule that is constructed by joining nucleic acid molecules and, in some embodiments, can replicate in a live cell. A “synthetic nucleic acid” refers to a molecule that is amplified or chemically, or by other means, synthesized. Synthetic nucleic acids include those that are chemically modified, or otherwise modified, but can base pair with naturally-occurring nucleic acid molecules. Recombinant nucleic acids and synthetic nucleic acids also include those molecules that result from the replication of either of the foregoing. Engineered nucleic acid of the present disclosure may be encoded by a single molecule (e.g., included in the same plasmid or other vector) or by multiple different molecules (e.g., multiple different independently-replicating molecules).

Engineered nucleic acid of the present disclosure may be produced using standard molecular biology methods (see, e.g., Green and Sambrook, Molecular Cloning, A Laboratory Manual, 2012, Cold Spring Harbor Press). In some embodiments, engineered nucleic acid constructs are produced using GIBSON ASSEMBLY® Cloning (see, e.g., Gibson, D. G. et al. Nature Methods, 343-345, 2009; and Gibson, D. G. et al. Nature Methods, 901-903, 2010, each of which is incorporated by reference herein). GIBSON ASSEMBLY® typically uses three enzymatic activities in a single-tube reaction: 5′ exonuclease, the ‘Y extension activity of a DNA polymerase and DNA ligase activity. The 5′ exonuclease activity chews back the 5′ end sequences and exposes the complementary sequence for annealing. The polymerase activity then fills in the gaps on the annealed regions. A DNA ligase then seals the nick and covalently links the DNA fragments together. The overlapping sequence of adjoining fragments is much longer than those used in Golden Gate Assembly, and therefore results in a higher percentage of correct assemblies. In some embodiments, engineered nucleic acid constructs are produced using IN-FUSION® cloning (Takara Bio USA).

A promoter refers to a control region of a nucleic acid sequence at which initiation and rate of transcription of the remainder of a nucleic acid sequence are controlled. A promoter may also contain sub-regions at which regulatory proteins and molecules may bind, such as RNA polymerase and other transcription factors. Promoters may be constitutive, inducible, activatable, repressible, tissue-specific or any combination thereof. A promoter drives expression or drives transcription of the nucleic acid sequence that it regulates. Herein, a promoter is considered to be operably linked when it is in a correct functional location and orientation in relation to a nucleic acid sequence it regulates to control (“drive”) transcriptional initiation and/or expression of that sequence.

Constitutive promoters are unregulated promoters that continually activate transcription. Non-limiting examples of constitutive promoters include the cytomegalovirus (CMV) promoter, the elongation factor 1-alpha (EFla) promoter, the elongation factor (EFS) promoter, the MND promoter (a synthetic promoter that contains the U3 region of a modified MoMuLV LTR with myeloproliferative sarcoma virus enhancer), the phosphoglycerate kinase (PGK) promoter, the spleen focus-forming virus (SFFV) promoter, the simian virus 40 (SV40) promoter, and the ubiquitin C (UbC) promoter.

Inducible promoters are promoters that are characterized by regulating (e.g., initiating or activating) transcriptional activity when in the presence of, influenced by or contacted by a signal. The signal may be endogenous or a normally exogenous condition (e.g., light), compound (e.g., chemical or non-chemical compound) or protein (e.g., cytokine) that contacts an inducible promoter in such a way as to be active in regulating transcriptional activity from the inducible promoter. Activation of transcription may involve directly acting on a promoter to drive transcription or indirectly acting on a promoter by inactivation a repressor that is preventing the promoter from driving transcription. Conversely, deactivation of transcription may involve directly acting on a promoter to prevent transcription or indirectly acting on a promoter by activating a repressor that then acts on the promoter. A promoter is considered responsive to a signal if in the presence of that signal transcription from the promoter is activated, deactivated, increased or decreased.

Also provided herein are vectors comprising the engineered genetic constructs of the present disclosure. In some embodiments, the vector is an episomal vector, such as a plasmid or viral vector (e.g., adenoviral vector, retroviral vector, herpes simplex virus vectors, and/or chimeric viral vectors).

### Cells

Engineered genetic constructs of the present disclosure may be delivered systemically and activated (transcription of the constructs are activated) conditionally (based on the presence or absence of input signals) in a particular target cell.

The difference between target cells and non-target cells may be, for example, disease state (e.g., disease v. non-disease), cell type (e.g., neuronal cell v. glial cell), or environmental state (e.g., T cell in a pro-inflammatory state v. T cell in an anti-inflammatory state). As provided herein, the choice of target cells (and non-target cell) is not limited to a particular type of cell or condition.

In some embodiments, a target cell is a cancerous cell, a benign tumor cell or other disease cell. Thus, in some embodiments, an engineered genetic construct is delivered to a subject having tumor cells or cancer cells, and the engineered genetic construct is expressed in the tumor cells or cancer cells.

A cancerous cell may be any type of cancerous cell, including, but not limited to, premalignant neoplasms, malignant tumors, metastases, or any disease or disorder characterized by uncontrolled cell growth such that it would be considered cancerous or precancerous. The cancer may be a primary or metastatic cancer. Cancers include, but are not limited to, ocular cancer, biliary tract cancer, bladder cancer, pleura cancer, stomach cancer, ovary cancer, meninges cancer, kidney cancer, brain cancer including glioblastomas and medulloblastomas, breast cancer, cervical cancer, choriocarcinoma, colon cancer, endometrial cancer, esophageal cancer, gastric cancer, hematological neoplasms including acute lymphocytic and myelogenous leukemia, multiple myeloma, AIDS-associated leukemias and adult T-cell leukemia lymphoma, intraepithelial neoplasms including Bowen's disease and Paget's disease, liver cancer, lung cancer, lymphomas including Hodgkin's disease and lymphocytic lymphomas, neuroblastomas, oral cancer including squamous cell carcinoma, ovarian cancer including those arising from epithelial cells, stromal cells, germ cells and mesenchymal cells, pancreatic cancer, prostate cancer, rectal cancer, sarcomas including leiomyosarcoma, rhabdomyosarcoma, liposarcoma, fibrosarcoma, and osteosarcoma, skin cancer including melanoma, Kaposi's sarcoma, basocellular cancer, and squamous cell cancer, testicular cancer including germinal tumors such as seminoma, non-seminoma, teratomas, choriocarcinomas, stromal tumors and germ cell tumors, thyroid cancer including thyroid adenocarcinoma and medullar carcinoma, and renal cancer including adenocarcinoma and Wilms' tumor. Commonly encountered cancers include breast, prostate, lung, ovarian, colorectal, and brain cancer. In some embodiments, the tumor is a melanoma, carcinoma, sarcoma, or lymphoma.

Engineered genetic constructs of the present disclosure may be expressed in a broad range of host cell types. In some embodiments, engineered genetic constructs are expressed in mammalian cells (e.g., human cells). Engineered genetic constructs of the present disclosure may be expressed in vivo, e.g., in a subject such as a human subject.

In some embodiments, engineered genetic constructs are expressed in mesenchymal stem cells (MSCs), induced pluripotent stem cells (iPSCs), embryonic stem cells (ESCs), natural killer (NK) cells, T cells, hematopoietic stem cells (HSCs), and/or other immune cells (e.g., for cells engineered ex vivo). In some embodiments, engineered genetic constructs are expressed in immune cells, muscle cells, liver cells, neurons, eye cells, ear cells, skin cells, heart cells, pancreatic cells, and/or fat cells (e.g., for cells targeted in vivo).

In some embodiments, engineered genetic constructs are expressed in mammalian cells. For example, in some embodiments, engineered genetic constructs are expressed in human cells, primate cells (e.g., vero cells), rat cells (e.g., GH3 cells, OC23 cells) or mouse cells (e.g., MC3T3 cells). There are a variety of human cell lines, including, without limitation, human embryonic kidney (HEK) cells, HeLa cells, cancer cells from the National Cancer Institute's 60 cancer cell lines (NCI60), DU145 (prostate cancer) cells, Lncap (prostate cancer) cells, MCF-7 (breast cancer) cells, MDA-MB-438 (breast cancer) cells, PC3 (prostate cancer) cells, T47D (breast cancer) cells, THP-1 (acute myeloid leukemia) cells, U87 (glioblastoma) cells, SHSY5Y human neuroblastoma cells (cloned from a myeloma) and Saos-2 (bone cancer) cells. In some embodiments, engineered nucleic acids are expressed in human embryonic kidney (HEK) cells (e.g., HEK 293 or HEK 293T cells). In some embodiments, engineered nucleic acids are expressed in stem cells (e.g., human stem cells) such as, for example, pluripotent stem cells (e.g., human pluripotent stem cells including human induced pluripotent stem cells (hiPSCs)). A “stem cell” refers to a cell with the ability to divide for indefinite periods in culture and to give rise to specialized cells. A “pluripotent stem cell” refers to a type of stem cell that is capable of differentiating into all tissues of an organism, but not alone capable of sustaining full organismal development. A “human induced pluripotent stem cell” refers to a somatic (e.g., mature or adult) cell that has been reprogrammed to an embryonic stem cell-like state by being forced to express genes and factors important for maintaining the defining properties of embryonic stem cells (see, e.g., Takahashi and Yamanaka, Cell 126 (4): 663-76, 2006, incorporated by reference herein). Human induced pluripotent stem cell cells express stem cell markers and are capable of generating cells characteristic of all three germ layers (ectoderm, endoderm, mesoderm).

Additional non-limiting examples of cell lines that may be used in accordance with the present disclosure include 293-T, 293-T, 3T3, 4T1, 721, 9L, A-549, A172, A20, A253, A2780, A2780ADR, A2780cis, A431, ALC, B16, B35, BCP-1, BEAS-2B, bEnd.3, BHK-21, BR 293, BxPC3, C2C12, C3H-10T1/2, C6, C6/36, Cal-27, CGR8, CHO, CML T1, CMT, COR-L23, COR-L23/5010, COR-L23/CPR, COR-L23/R23, COS-7, COV-434, CT26, D17, DH82, DU145, DuCaP, E14Tg2a, EL4, EM2, EM3, EMT6/AR1, EMT6/AR10.0, FM3, H1299, H69, HB54, HB55, HCA2, Hepalclc7, High Five cells, HL-60, HMEC, HT-29, HUVEC, J558L cells, Jurkat, JY cells, K562 cells, KCL22, KG1, Ku812, KYO1, LNCap, Ma-Mel 1, 2, 3 . . . 48, MC-38, MCF-10A, MCF-7, MDA-MB-231, MDA-MB-435, MDA-MB-468, MDCK II, MG63, MONO-MAC 6, MOR/0.2R, MRC5, MTD-1A, MyEnd, NALM-1, NCI-H69/CPR, NCI-H69/LX10, NCI-H69/LX20, NCI-H69/LX4, NIH-3T3, NW-145, OPCN/OPCT Peer, PNT-1A/PNT 2, PTK2, Raji, RBL cells, RenCa, RIN-5F, RMA/RMAS, S2, Saos-2 cells, Sf21, Sf9, SiHa, SKBR3, SKOV-3, T-47D, T2, T84, THP1, U373, U87, U937, VCaP, WM39, WT-49, X63, YAC-1 and YAR cells.

### Compositions and Kits

The present disclosure also provides compositions comprising an engineered genetic construct comprising at least one synthetic promoter that has higher activity in target cells relative to non-target cells and is operably linked to (a) a nucleotide sequence encoding a product of interest and (b) a 3′ untranslated region (UTR) comprising at least one microRNA (miRNA) sensor that includes at least one miRNA binding site to which at least one miRNA binds, wherein the at least one miRNA is inactive or active at a low level in the target cells, and wherein the at least one miRNA is active in non-target cells at a level detectable by the miRNA sensor.

The present disclosure further provides kits comprising an engineered genetic construct comprising at least one synthetic promoter that has higher activity in target cells relative to non-target cells and is operably linked to a 3′ untranslated region (UTR) comprising at least one microRNA (miRNA) sensor that includes at least one miRNA binding site to which at least one miRNA binds, wherein the at least one miRNA is inactive or active at a low level in the target cells, and wherein the at least one miRNA is active in non-target cells at a level detectable by the miRNA sensor, wherein the construct further comprises restriction sites located between the promoter and the 3′ UTR.

A composition and/or kit of the present disclosure may include any of the engineered genetic constructs, including any of the synthetic promoters and/or miRNA sensors, as described herein.

### Methods

Also provided herein are methods comprising delivering to a cell an engineered genetic construct comprising at least one synthetic promoter that has higher activity in target cells relative to non-target cells and is operably linked to (a) a nucleotide sequence encoding a product of interest and (b) a 3′ untranslated region (UTR) comprising at least one microRNA (miRNA) sensor that includes at least one miRNA binding site to which at least one miRNA binds, wherein the at least one miRNA is not expressed in the target cells or is expressed in the target cells at a level undetectable by the miRNA sensor, and wherein the at least one miRNA is expressed in non-target cells at a level detectable by the miRNA sensor (such that the engineered genetic construct is expressed in target cells and silenced and/or degraded in non-target cells).

Vectors comprising the engineered genetic construct may also be delivered to a cell, in some embodiments.

Further still, the present disclosure provides delivering to a subject an engineered genetic construct comprising at least one synthetic promoter that has higher activity in target cells relative to non-target cells and is operably linked to (a) a nucleotide sequence encoding a product of interest and (b) a 3′ untranslated region (UTR) comprising at least one microRNA (miRNA) sensor that includes at least one miRNA binding site to which at least one miRNA binds, wherein the at least one miRNA is not expressed in the target cells or is expressed in the target cells at a level undetectable by the miRNA sensor, and wherein the at least one miRNA is expressed in non-target cells at a level detectable by the miRNA sensor.

Vectors comprising the engineered genetic construct may also be delivered to a subject, in some embodiments.

In some embodiments, a subject is a mammalian subject. In some embodiments, a subject is a human subject.

Methods of the present disclosure may include (use of) any of the engineered genetic constructs, including any of the synthetic promoters and/or miRNA sensors, as described herein.

Engineered genetic constructs may be delivered to cells using a viral delivery system (e.g., retroviral, adenoviral, adeno-association, helper-dependent adenoviral systems, hybrid adenoviral systems, herpes simplex, pox virus, lentivirus, Epstein-Barr virus) or a non-viral delivery system (e.g., physical: naked DNA, DNA bombardment, electroporation, hydrodynamic, ultrasound or magnetofection; or chemical: cationic lipids, different cationic polymers or lipid polymer) (Nayerossadat N et al. Adv Biomed Res. 2012; 1: 27, incorporated herein by reference). In some embodiments, the non-viral based deliver system is a hydrogel-based delivery system (see, e.g., Brandl F, et al. Journal of Controlled Release, 2010, 142(2): 221-228, incorporated herein by reference).

Engineered genetic constructs and/or cells may be delivered to a subject (e.g., a mammalian subject, such as a human subject) by any in vivo delivery method known in the art. For example, engineered genetic constructs and/or cells may be delivered intravenously. In some embodiments, engineered genetic constructs and/or cells are delivered in a delivery vehicle (e.g., non-liposomal nanoparticle or liposome). In some embodiments, engineered genetic constructs and/or cells are delivered systemically to a subject having a cancer or other disease and activated (transcription is activated) specifically in cancer cells or diseased cells of the subject.

### Additional Embodiments

1. A synthetic genetic circuit (construct) that achieves spatial and/or temporal selectivity comprising one or more artificial promoters that are active in specific cell types, but not others, is/are operably linked to and drive expression of one or more nucleic acid molecules, further comprising an miRNA sensor component that detects one or more miRNAs that are downregulated in specific on-target cells, but not in off-target cells, in which miRNA-based suppression of expression the nucleic acid molecules is carried out in off-target cells, such that expression of nucleic acid molecules is reduced in off-target cells compared with on-target cells.
2. The synthetic genetic circuit of embodiment 1, wherein the one or more artificial promoters is/are operably linked to and drive expression of one or more nucleic acid molecules encoding therapeutic or marker polypeptides.
3. The synthetic genetic circuit embodiment 1 or embodiment 2, wherein each of the one or more artificial promoters comprises between 200-300 base pairs.
4. The synthetic genetic circuit of any one of embodiments 1, wherein the one or more artificial promoters comprises at least 10-fold enhanced activity within on-target cells versus off-target cells, preferably at least 50-fold enhanced activity within on-target cells versus off-target cells.
5. The synthetic genetic circuit of any one of embodiments 1-4, wherein the synthetic genetic circuit comprises multiple microRNA sensors encoded in tandem on the 3′end of target transcripts such that high-level gene expression is allowed only when multiple microRNAs are absent in on-target cells.

### EXAMPLES

### Example 1

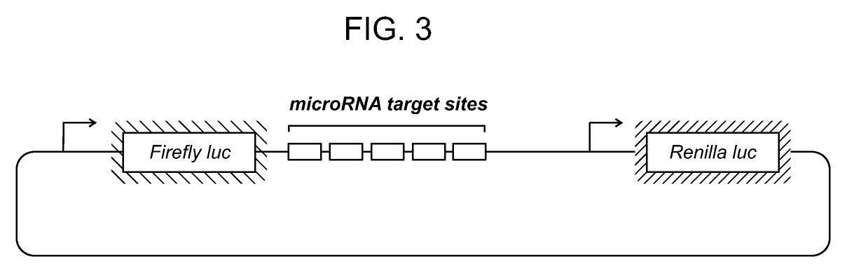

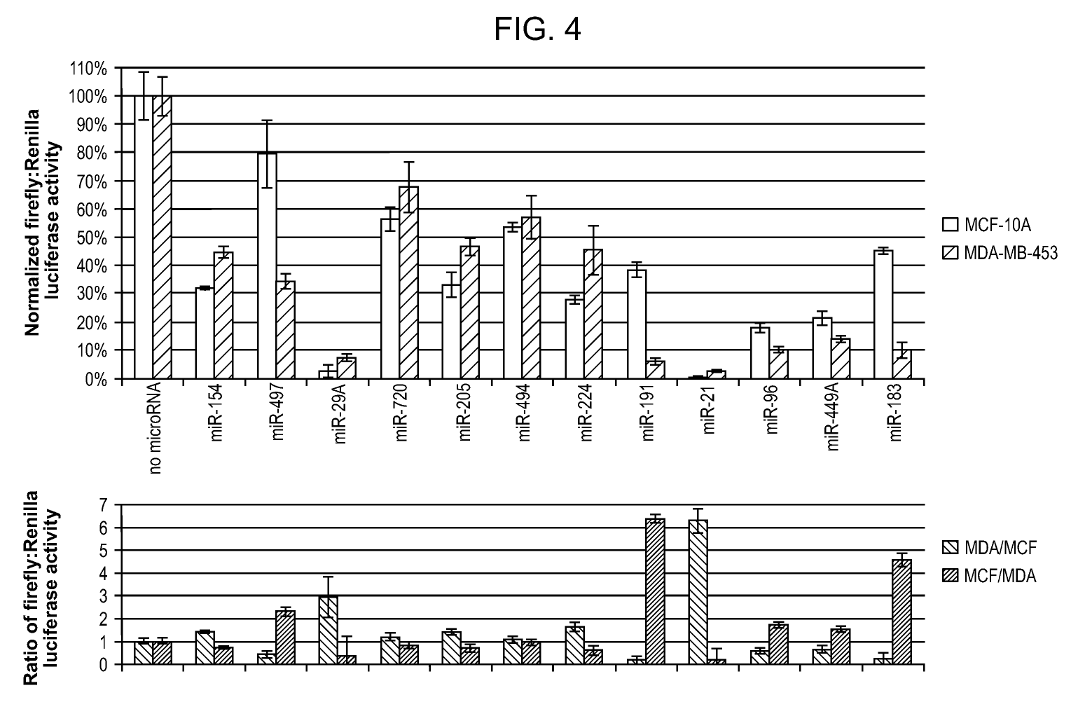

In this Example, five copies of microRNA target sites were cloned into the 3′-UTR of firefly luciferase: microRNA target sites designated 154, 497, 29A, 720, 205, 494, 224, 191, 21, 96, 449A, or 183. See FIG. 3. Firefly luciferase served as the experimental reporter, and Renilla luciferase served as the control reporter. Normalization of the firefly to Renilla luciferase expression helps control for transfection efficiencies and nonspecific cellular responses. The plasmids carrying the microRNA target sites and reporters were transfected into MCF-10A and MDA-MB-453 cell lines and luciferase expression was measured the following day. microRNAs inhibited expression of the reporter gene at different levels for the two cell lines. See FIG. 4, top panel. Certain microRNAs (miR-191, miR-21 and miR-183) had greater than 5-fold selectivity between the cell types. See FIG. 4, bottom panel.

### Example 2

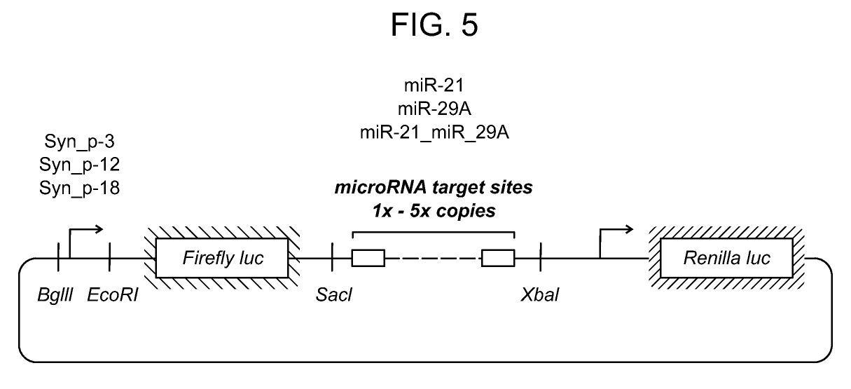

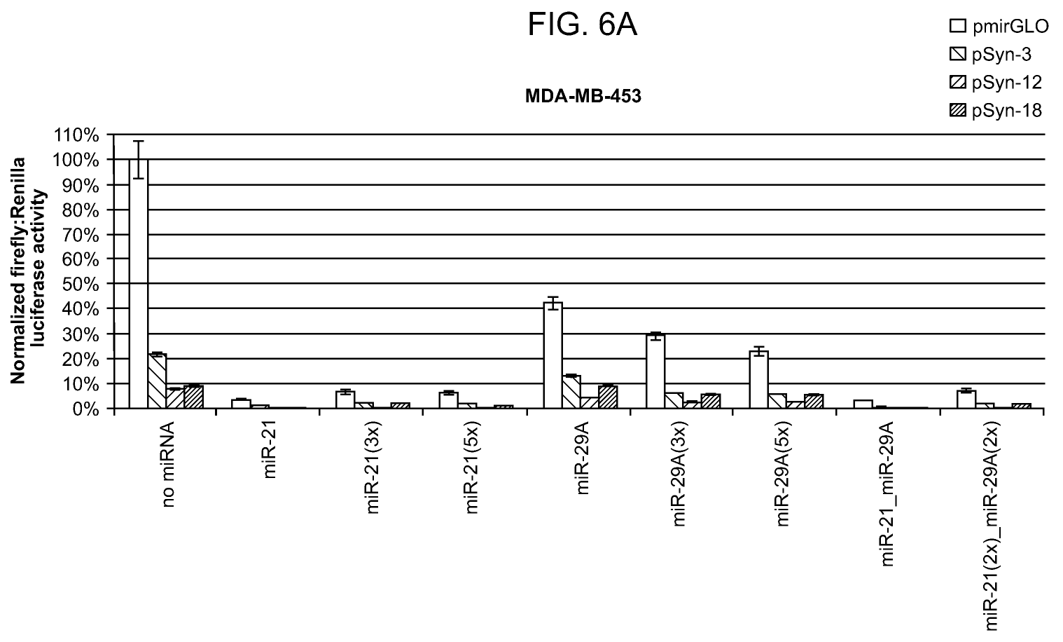

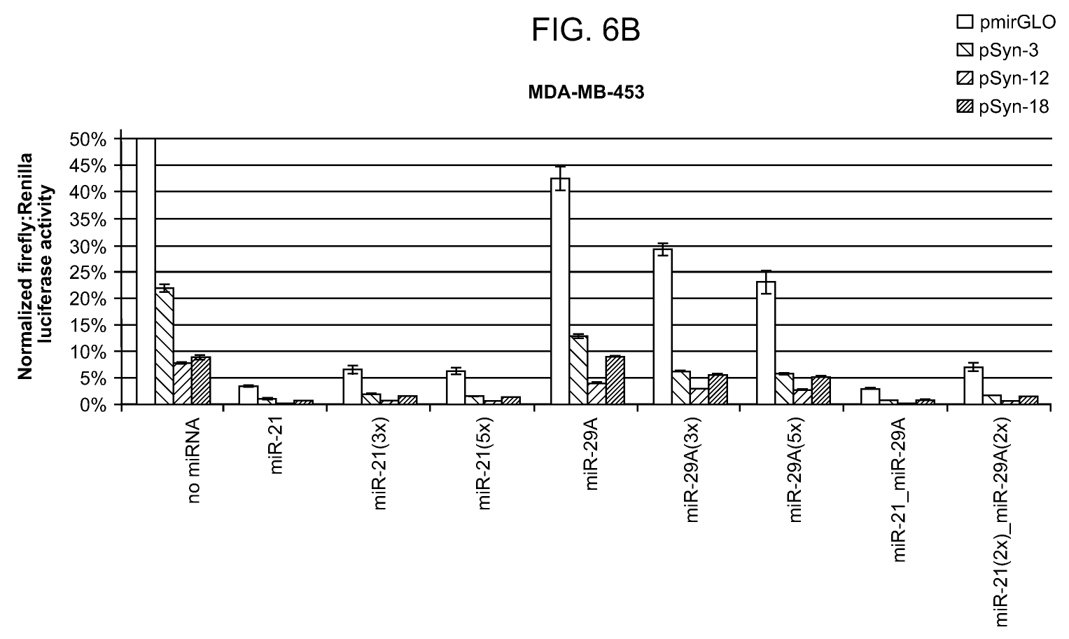

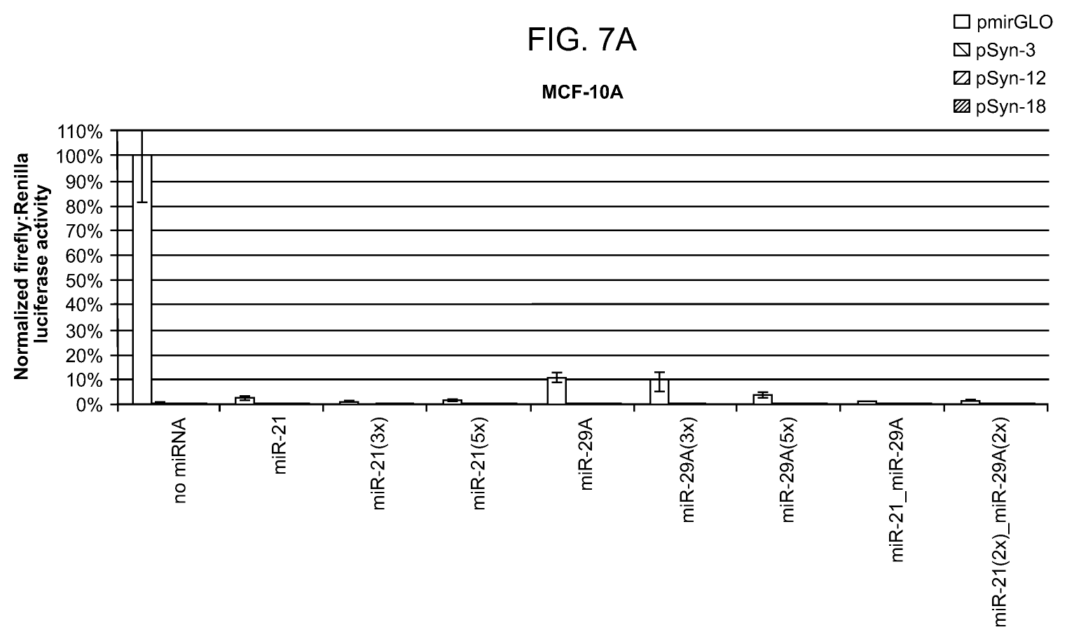

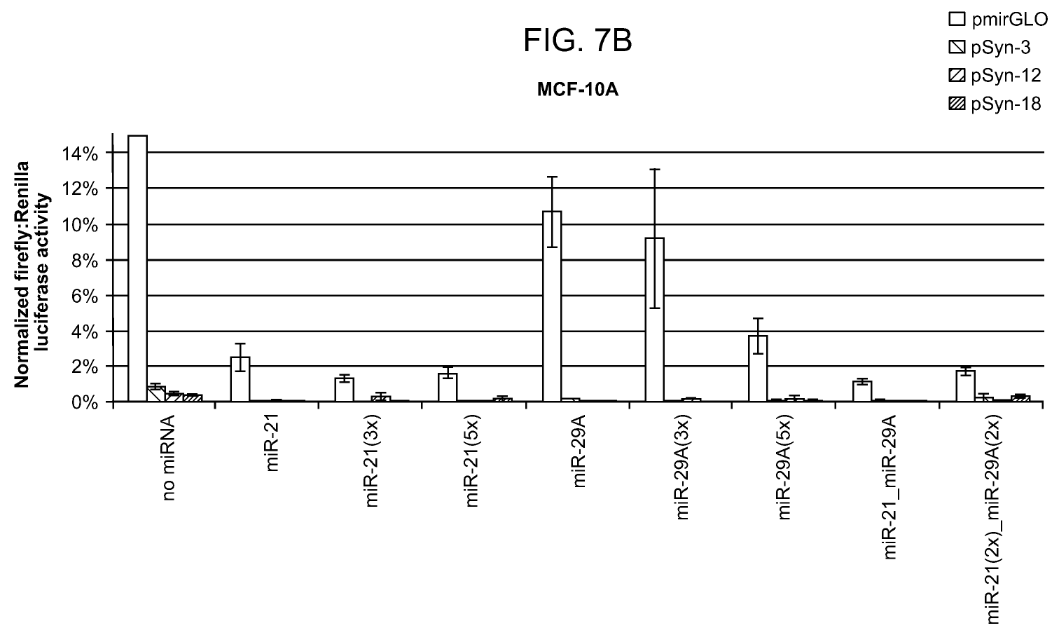

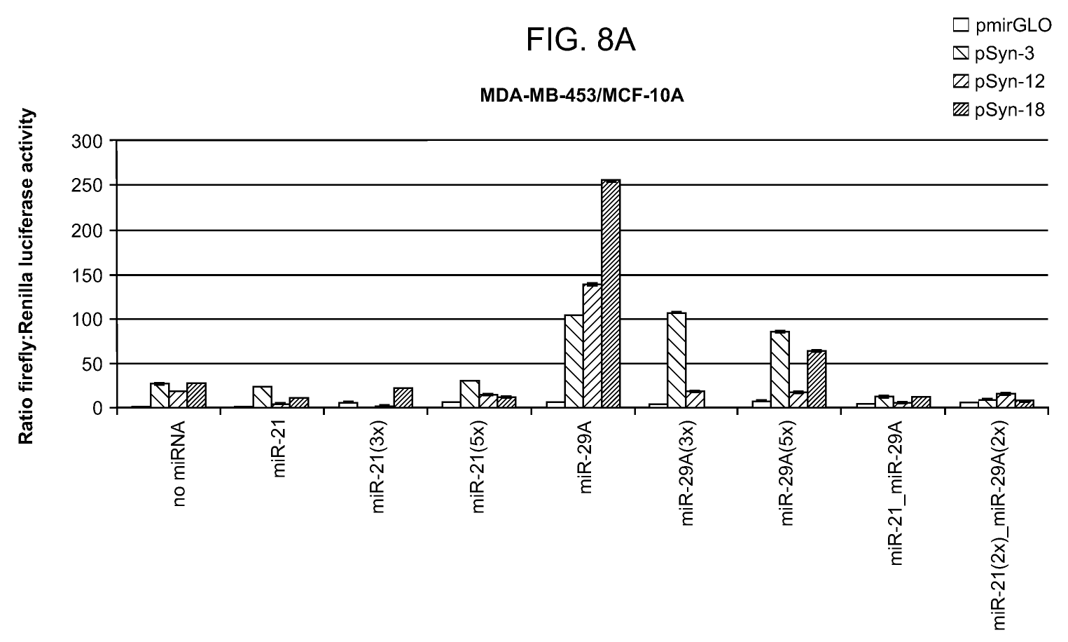

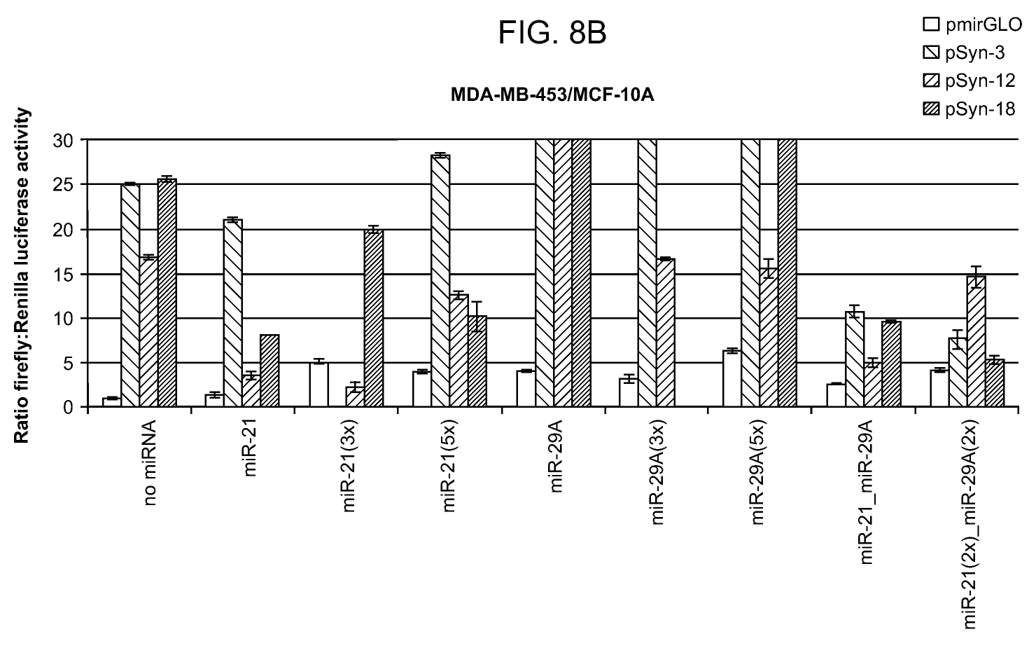

In this example, three different synthetic promoters were assayed in combination with two different microRNA target sites (1-5 copies). See FIG. 5. The expression data are shown in FIGS. 6A-8B. A number of combinations exhibited greater than 50-fold selectivity for malignant vs. non-malignant cell lines. An unexpected synergistic effect was observed in several of the spatiotemporal regulators constructs containing both a synthetic promoter and a microRNA sensor. For example, constructs containing only synthetic promoters pSyn-3, 12, 18 (without a miRNA sensor) exhibited cell selectivity of 25× (ratio of reporter expression in MDA-MB-453/MCF-10A cells), while constructs containing only miRNA-29A (without a synthetic promoter) exhibited cell selectivity of 4×. Spatiotemporal regulator constructs that include both (1) pSyn-3, pSyn-12, or pSyn-18 and (2) miRNA-29A exhibits cell selectivity of ˜100×, ˜150× and ˜250×, respectively.

### REFERENCES

1. Frakes A E, Ferraiuolo L, Haidet-Phillips A M, Schmelzer L, Braun L, Miranda C J, Ladner K J, Bevan A K, Foust K D, Godbout J P, Popovich P G, Guttridge D C, Kaspar B K. Microglia induce motor neuron death via the classical NF-kappaB pathway in amyotrophic lateral sclerosis. Neuron. 2014; 81(5): 1009-23. doi: 10.1016/j.neuron.2014.01.013. PubMed PMID: 24607225; PMCID: PMC3978641.
2. Xie Z, Wroblewska L, Prochazka L, Weiss R, Benenson Y. Multi-input RNAi-based logic circuit for identification of specific cancer cells. Science. 2011; 333(6047):1307-11. Epub 2011/09/03. doi: 10.1126/science.1205527. PubMed PMID: 21885784.
3. Saxena P, Heng B C, Bai P, Folcher M, Zulewski H, Fussenegger M. A programmable synthetic lineage-control network that differentiates human IPSCs into glucose-sensitive insulin-secreting beta-like cells. Nature communications. 2016; 7:11247. doi: 10.1038/ncomms11247. PubMed PMID: 27063289; PMCID: PMC4831023.
4. Rogakou E P, Pilch D R, Orr A H, Ivanova V S, Bonner W M. DNA double-stranded breaks induce histone H2AX phosphorylation on serine 139. J Biol Chem. 1998; 273(10):5858-68. PubMed PMID: 9488723.
5. Barretina J, Caponigro G, Stransky N, Venkatesan K, Margolin A A, Kim S, Wilson C J, Lehar J, Kryukov G V, Sonkin D, Reddy A, Liu M, Murray L, Berger M F, Monahan J E, Morais P, Meltzer J, Korejwa A, Jane-Valbuena J, Mapa F A, Thibault J, Bric-Furlong E, Raman P, Shipway A, Engels I H, Cheng J, Yu G K, Yu J, Aspesi P, Jr., de Silva M, Jagtap K, Jones M D, Wang L, Hatton C, Palescandolo E, Gupta S, Mahan S, Sougnez C, Onofrio R C, Liefeld T, MacConaill L, Winckler W, Reich M, Li N, Mesirov J P, Gabriel S B, Getz G, Ardlie K, Chan V, Myer V E, Weber B L, Porter J, Warmuth M, Finan P, Harris J L, Meyerson M, Golub T R, Morrissey M P, Sellers W R, Schlegel R, Garraway L A. The Cancer Cell Line Encyclopedia enables predictive modelling of anticancer drug sensitivity. Nature. 2012; 483(7391):603-7. doi: 10.1038/nature11003. PubMed PMID: 22460905; PMCID: PMC3320027.
6. Huang W Y, Hsu S D, Huang H Y, Sun Y M, Chou C H, Weng S L, Huang H D. MethHC: a database of DNA methylation and gene expression in human cancer. Nucleic Acids Res. 2015; 43(Database issue):D856-61. doi: 10.1093/nar/gku1151. PubMed PMID: 25398901; PMCID: PMC4383953.
7. Gure A O, Tureci O, Sahin U, Tsang S, Scanlan M J, Jager E, Knuth A, Pfreundschuh M, Old L J, Chen Y T. SSX: a multigene family with several members transcribed in normal testis and human cancer. Int J Cancer. 1997; 72(6):965-71. PubMed PMID: 9378559.
8. Landgraf P, Rusu M, Sheridan R, Sewer A, Iovino N, Aravin A, Pfeffer S, Rice A, Kamphorst A O, Landthaler M, Lin C, Socci N D, Hermida L, Fulci V, Chiaretti S, Foa R, Schliwka J, Fuchs U, Novosel A, Muller R U, Schermer B, Bissels U, Inman J, Phan Q, Chien M, Weir D B, Choksi R, De Vita G, Frezzetti D, Trompeter H I, Hornung V, Teng G, Hartmann G, Palkovits M, Di Lauro R, Wernet P, Macino G, Rogler C E, Nagle J W, Ju J, Papavasiliou F N, Benzing T, Lichter P, Tam W, Brownstein M J, Bosio A, Borkhardt A, Russo J J, Sander C, Zavolan M, Tuschl T. A mammalian microRNA expression atlas based on small RNA library sequencing. Cell. 2007; 129(7):1401-14. doi: 10.1016/j.cell.2007.04.040. PubMed PMID: 17604727; PMCID: PMC2681231.
9. Gitzinger M, Kemmer C, El-Baba M D, Weber W, Fussenegger M. Controlling transgene expression in subcutaneous implants using a skin lotion containing the apple metabolite phloretin. Proc Natl Acad Sci USA. 2009; 106(26):10638-43. doi: 10.1073/pnas.0901501106. PubMed PMID: 19549857; PMCID: PMC2700147.
10. Fux C, Moser S, Schlatter S, Rimann M, Bailey J E, Fussenegger M. Streptogramin- and tetracycline-responsive dual regulated expression of p27Kip1 sense and antisense enables positive and negative growth control of Chinese hamster ovary cells. Nucleic Acids Research. 2001; 29(4):e19. doi: 10.1093/nar/29.4.e19.
11. Fussenegger M, Morris R P, Fux C, Rimann M, von Stockar B, Thompson C J, Bailey J E. Streptogramin-based gene regulation systems for mammalian cells. Nat Biotech. 2000; 18(11):1203-8.
12. Weber W, Fux C, Daoud-El Baba M, Keller B, Weber C C, Kramer B P, Heinzen C, Aubel D, Bailey J E, Fussenegger M. Macrolide-based transgene control in mammalian cells and mice. Nat Biotech. 2002; 20(9):901-7. doi: http://www.nature.com/nbt/journal/v20/n9/suppinfo/nbt731_S1.html.
13. Gitzinger M, Kemmer C, Fluri D A, El-Baba M D, Weber W, Fussenegger M. The food additive vanillic acid controls transgene expression in mammalian cells and mice. Nucleic Acids Res. 2012; 40(5):e37. doi: 10.1093/nar/gkr1251. PubMed PMID: 22187155; PMCID: PMC3300003.
14. Hirrlinger J, Requardt R P, Winkler U, Wilhelm F, Schulze C, Hirrlinger P G. Split-CreERT2: temporal control of DNA recombination mediated by split-Cre protein fragment complementation. PLoS One. 2009; 4(12):e8354. doi: 10.1371/journal.pone.0008354. PubMed PMID: 20016782; PMCID: PMC2791205.
15. Feil R, Wagner J, Metzger D, Chambon P. Regulation of Cre recombinase activity by mutated estrogen receptor ligand-binding domains. Biochem Biophys Res Commun. 1997; 237(3):752-7. doi: 10.1006/bbrc.1997.7124. PubMed PMID: 9299439.
16. Sharma N, Moldt B, Dalsgaard T, Jensen T G, Mikkelsen J G. Regulated gene insertion by steroid-induced PhiC31 integrase. Nucleic Acids Res. 2008; 36(11):e67. doi: 10.1093/nar/gkn298. PubMed PMID: 18499713; PMCID: PMC2441784.
17. Hunter N L, Awatramani R B, Farley F W, Dymecki S M. Ligand-activated Flpe for temporally regulated gene modifications. Genesis. 2005; 41(3):99-109. doi: 10.1002/gene.20101. PubMed PMID: 15729687.
18. Jullien N, Sampieri F, Enjalbert A, Herman J P. Regulation of Cre recombinase by ligand-induced complementation of inactive fragments. Nucleic Acids Res. 2003; 31(21):e131. PubMed PMID: 14576331; PMCID: PMC275488.
19. Yang L, Nielsen A A, Fernandez-Rodriguez J, McClune C J, Laub M T, Lu T K, Voigt C A. Permanent genetic memory with >1-byte capacity. Nat Methods. 2014. doi: 10.1038/nmeth.3147. PubMed PMID: 25344638.
20. Canver M C, Bauer D E, Dass A, Yien Y Y, Chung J, Masuda T, Maeda T, Paw B H, Orkin S H. Characterization of genomic deletion efficiency mediated by clustered regularly interspaced palindromic repeats (CRISPR)/Cas9 nuclease system in mammalian cells. J Biol Chem. 2014; 289(31):21312-24. doi: 10.1074/jbc.M114.564625. PubMed PMID: 24907273; PMCID: PMC4118095.
21. Negroni L, Samson M, Guigonis J-M, Rossi B, Pierrefite-Carle V, Baudoin C. Treatment of colon cancer cells using the cytosine deaminase/5-fluorocytosine suicide system induces apoptosis, modulation of the proteome, and Hsp90β phosphorylation. Molecular Cancer Therapeutics. 2007; 6(10):2747-56. doi: 10.1158/1535-7163.mct-07-0040.
22. Kievit E, Bershad E, Ng E, Sethna P, Dev I, Lawrence T S, Rehemtulla A. Superiority of yeast over bacterial cytosine deaminase for enzyme/prodrug gene therapy in colon cancer xenografts. Cancer Res. 1999; 59(7):1417-21. PubMed PMID: 10197605.
23. Beck C, Cayeux S, Lupton S D, Dorken B, Blankenstein T. The thymidine kinase/ganciclovir-mediated “suicide” effect is variable in different tumor cells. Human gene therapy. 1995; 6(12): 1525-30. doi: 10.1089/hum. 1995.6.12-1525. PubMed PMID: 8664377.
24. Deans T L, Cantor C R, Collins J J. A tunable genetic switch based on RNAi and repressor proteins for regulating gene expression in mammalian cells. Cell. 2007; 130(2):363-72. Epub 2007/07/31. doi: S0092-8674(07)00684-8 [pii]10.1016/j.cell.2007.05.045. PubMed PMID: 17662949.
25. Wroblewska L, Kitada T, Endo K, Siciliano V, Stillo B, Saito H, Weiss R. Mammalian synthetic circuits with RNA binding proteins for RNA-only delivery. Nat Biotechnol. 2015; 33(8):839-41. doi: 10.1038/nbt.3301. PubMed PMID: 26237515; PMCID: PMC4532950.
26. Muller P Y, Milton M N. The determination and interpretation of the therapeutic index in drug development. Nat Rev Drug Discov. 2012; 11(10):751-61. doi: 10.1038/nrd3801. PubMed PMID: 22935759.
27. Capelli C, Pedrini O, Valgardsdottir R, Da Roit F, Golay J, Introna M. Clinical grade expansion of MSCs. Immunol Lett. 2015; 168(2):222-7. doi: 10.1016/j.imlet.2015.06.006. PubMed PMID: 26092523.

All references, patents and patent applications disclosed herein are incorporated by reference with respect to the subject matter for which each is cited, which in some cases may encompass the entirety of the document.

The indefinite articles “a” and “an,” as used herein in the specification and in the claims, unless clearly indicated to the contrary, should be understood to mean “at least one.”

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

What is claimed is:

1. An engineered genetic construct comprising at least one synthetic promoter that has higher activity in target cells relative to non-target cells and is operably linked to (a) a nucleotide sequence encoding a product of interest and (b) a 3′ untranslated region (UTR) comprising at least one microRNA (miRNA) sensor that includes at least one miRNA binding site to which at least one miRNA binds, wherein the at least one miRNA is inactive or active at a low level in the target cells, and wherein the at least one miRNA is active in non-target cells at a level detectable by the miRNA sensor.

2. The engineered genetic construct of claim 1, wherein the microRNA sensor includes at least two miRNA binding sites.

3. The engineered genetic construct of claim 2, wherein the microRNA sensor includes 2-10 miRNA binding sites.

4. The engineered genetic construct of claim 3, wherein the microRNA sensor includes 5-10 miRNA binding sites.

5. The engineered genetic construct of claim 2, wherein the microRNA sensor includes at least five miRNA binding sites.

6. The engineered genetic construct of claim 5, wherein the microRNA sensor includes 5-10 miRNA binding sites.

7. The engineered genetic construct of any one of claims 2-6, wherein the miRNA binding sites are located in tandem.

8. The engineered genetic construct of any one of claims 2-7, wherein the miRNA binding sites are identical to each other.

9. The engineered genetic construct of any one of claims 1-8, wherein the 3′ UTR comprising at least two miRNA sensors, each specific to a different miRNA.

10. The engineered genetic construct of any one of claims 1-9, wherein the at least one miRNA (miR) is selected from miR-154, miR-497, miR-29A, miR-720, miR-205, miR-494, miR-224, miR-191, miR-21, miR-96, miR-449A and miR-183.

11. The engineered genetic construct of any one of claims 1-10, wherein the target cells are cancerous cells, immune cells, or neurons.

12. The engineered genetic construct of any one of claims 1-11, wherein the synthetic promoter has a length of 100-500 nucleotides.

13. The engineered genetic construct of claim 12, wherein the synthetic promoter has a length of 100-125 nucleotides.

14. The engineered genetic construct of any one of claims 1-13, wherein the synthetic promoter includes tandem repeat nucleotide sequences.

15. The engineered genetic construct of claim 14, wherein the length of each of the nucleotide sequences is less than 12 nucleotides.

16. The engineered genetic construct of claim 14 or 15, wherein the synthetic promoter includes 2-20 tandem repeat nucleotide sequences.

17. The engineered genetic construct of any one of claims 1-16, wherein a nucleotide spacer is positioned between each of the repeat nucleotides sequences.

18. The engineered genetic construct of any one of claims 1-17, wherein the activity of the synthetic promoter is at least 10% higher in the target cells relative to non-target cells.

19. The engineered genetic construct of claim 18, wherein the activity of the synthetic promoter is at least 50% higher in the target cells relative to non-target cells.

20. The engineered genetic construct of claim 19, wherein the activity of the synthetic promoter is at least 100% higher in the target cells relative to non-target cells.

21. The engineered genetic construct of any one of claims 1-19, wherein the product of interest is a therapeutic molecule, a prophylactic molecule and/or a diagnostic molecule.

22. The engineered genetic construct of any one of claims 1-21, wherein the product of interest is a protein, peptide or nucleic acid.

23. The engineered genetic construct of claim 22, wherein the product of interest is a nucleic acid selected from RNA, DNA or a combination of RNA and DNA.

24. The engineered genetic construct of claim 23, wherein product of interest is a RNA selected from short-hairpin RNAs, short interfering RNAs and micro RNAs.

25. The engineered genetic construct of claim 21, wherein the product of interest is a therapeutic and/or prophylactic molecule selected from antibodies, enzymes, hormones, inflammatory molecules, anti-inflammatory molecules, immunomodulatory molecules, and anti-cancer molecules.

26. The engineered genetic construct of claim 21, wherein the product of interest is a diagnostic molecule selected from fluorescent molecules and luminescent molecules.

27. A vector comprising the engineered genetic construct of any one of claims 1-26.

28. A cell comprising the engineered genetic construct of any one of claims 1-26 or the vector of claim 27.

29. A composition comprising the engineered genetic construct of any one of claims 1-26, the vector of claim 27, or the cell of claim 28.

30. A kit comprising an engineered genetic construct comprising at least one synthetic promoter that has higher activity in target cells relative to non-target cells and is operably linked to a 3′ untranslated region (UTR) comprising at least one microRNA (miRNA) sensor that includes at least one miRNA binding site to which at least one miRNA binds, wherein the at least one miRNA is inactive or active at a low level in the target cells, and wherein the at least one miRNA is active in non-target cells at a level detectable by the miRNA sensor, wherein the construct further comprises restriction sites located between the promoter and the 3′ UTR.

31. A method comprising delivery to a cell the engineered genetic construct of any one of claims 1-26 or the vector of claim 27.

32. A method comprising delivery to a subject the engineered nucleic acid of any one of claims 1-26 or the vector of claim 26.

33. The method of claim 31, wherein the subject is a human subject.
